(19) United States
(12) Patent Application Publication
(10) Pub. No.: US 2021/0015733 A1
(43) Pub. Date: Jan. 21, 2021

(54) AEROGELS AND THEIR USE IN COSMETIC APPLICATIONS

(71) Applicant: BASF SE, Ludwigshafen (DE)

(72) Inventors: Volker Wendel, Düsseldorf-Holthausen (DE); Wibke Loelsberg, Ludwigshafen (DE); David Graham, Düsseldorf-Holthausen (DE); Daniel Stadler, Berlin (DE); Anja Suckert, Düsseldorf-Holthausen (DE); Heidi Riedel, Düsseldorf-Holthausen (DE); Nathalie Bouillo, Ludwigshafen (DE); Marc Fricke, Lemförde (DE); Claudia Wood, Ludwigshafen (DE)

(21) Appl. No.: 15/733,701

(22) PCT Filed: Apr. 1, 2019
(86) PCT No.: PCT/EP2019/058129

§ 371 Date: Sep. 30, 2020

(30) Foreign Application Priority Data
Apr. 9, 2018 (CN) PCT/CN2018/082353
May 4, 2018 (EP) 18170783.7

(51) Int. Cl. A61K 8/73; A61K 8/02; A61K 8/04; A61Q 19/00

(52) U.S. Cl. CPC A61K 8/733; A61K 8/0279; A61K 2800/40; A61Q 19/00; A61K 2800/10; A61K 8/042

(57) ABSTRACT

The present invention relates to a cosmetic and/or dermatological composition comprising, in a physiologically acceptable medium aerogel particles comprising an alginate, as well as the cosmetic use of a cosmetic and/or dermatological composition comprising, in a physiologically acceptable medium aerogel particles comprising an alginate, for treating the skin.

The present invention relates to a cosmetic and/or dermatological composition comprising, in a physiologically acceptable medium aerogel particles comprising an alginate, as well as the cosmetic use of a cosmetic and/or dermatological composition comprising, in a physiologically acceptable medium aerogel particles comprising an alginate, for treating the skin.

For the purposes of the present invention, an aerogel is a porous material which has been produced by a sol-gel process in which the liquid phase has been removed from the gel under supercritical conditions. The use of aerogels and alginates in cosmetic compositions in principle is known from the state of the art. Aerogels are ultra-light porous materials. The first aerogels were made by Kristler in 1932. They are generally synthesized via a sol-gel process in a liquid medium and then dried, usually by extraction with a supercritical fluid, the one most commonly used being supercritical CO2. This type of drying makes it possible to avoid shrinkage of the pores and of the material. Other types of drying also make it possible to obtain porous materials starting from gel, namely cryodesiccation, which consists in solidifying the gel at low temperature and in then subliming the solvent, and drying by evaporation. The materials thus obtained are referred to respectively as cryogels and xerogels.

For example WO 2013/190104 A2 discloses cosmetic and/or dermatological composition comprising, hydrophobic aerogel particles in a physiologically acceptable medium, silica and at least one particle that absorbs sebum. It also relates to a process for caring for and/or making up the skin, comprising the topical application of the composition to the skin. WO 2013/190104 A2 further discloses a cosmetic process for making the skin matt and/or for reducing its shine, comprising the topical application to the skin of the composition.

US 2015/0190319 A1 relates to a cosmetic composition, in particular a hair composition, comprising a combination of hydrophobic silica aerogel particles and particular thickening polymers, and also to a method of using said composition.

Depending on the form of application, cosmetic compositions may comprise different constituents in order to modify and optimize the properties of the composition.

The compositions disclosed in the state of the art generally are suitable for absorption and retention of bodily secretions e.g. sebum. There is a need for compositions which are suitable for release or absorption and release of formulation components such as emollients, or active materials.

One object of the present invention was to provide compositions which can be applied to the skin to modify the sensory effect. A further object of the present invention was to provide compositions which might be used for delivery of active components, preferably compositions which can be used for controlled release of such active components. Finally it was an object of the present invention to provide compositions which comprise biodegradable components.

This object has been solved by a cosmetic and/or dermatological composition comprising, in a physiologically acceptable medium aerogel particles comprising an alginate.

In the context of the present invention, the term “aerogels particles comprising an alginate” means that the aerogel comprises an alginate as part of the aerogel structure. Aerogels particles comprising an alginate are in principle known from the state of the art and are for example disclosed in WO 2015/177081 A1.

It has surprisingly been found that the use of aerogel particles comprising an alginate in cosmetic and/or dermatological compositions results in a composition which results in a positive sensory effect when applied to the skin. Furthermore, film formation could be observed. The compositions according to the present invention also can be used for delivery of active components, in particular for controlled release of said active components.

The composition according to the invention may be cosmetic and/or dermatological, preferably cosmetic. It was surprisingly found that stable compositions based on water or oil could be obtained with the aerogel particles comprising an alginate. The composition according to the invention may be in any pharmaceutical form conventionally used for a topical application and especially in the form of dispersions of gel or lotion type, emulsions of liquid or semiliquid consistency of the milk type, obtained by dispersing an oil phase in an aqueous phase (O/W) or vice versa (W/O), or suspensions or emulsions of soft, semi-solid or solid consistency of the cream or gel type, or alternatively multiple emulsions (W/O/W or O/W/O), microemulsions, vesicular dispersions of ionic and/or non-ionic type, or wax/aqueous phase dispersions.

In addition, the compositions used according to the invention can be more or less fluid and can have the appearance of a white or colored cream, of an ointment, of a milk, of a lotion, of a serum, of a paste or of a foam. They may optionally be applied to the skin in the form of an aerosol. They can also be in solid form, for example in the form of a stick.

The composition according to the invention is generally suitable for topical application to the skin and thus generally comprises a physiologically acceptable medium, i.e. a medium that is compatible with the skin and/or its integuments. It is preferably a cosmetically acceptable medium, i.e. a medium which has a pleasant color, odor and feel and which does not cause any unacceptable discomfort (stinging, tautness or redness) liable to discourage the consumer from using this composition.

Suitable aerogel particles are in principle known to the person skilled in the art. Suitable aerogel particles preferably form stable compositions with the additional components of the composition.

Suitable aerogel particles preferably have a bulk density in the range of from 0.03 to 1 kg/m³, more preferable in the range of from 0.04 to 0.8 kg/m³, in particular in the range of from 0.05 to 0.5 kg/m³. In the context of the present invention, the bulk density is determined using the DIN 53420 method for powdery products unless otherwise stated.

Therefore, according to a further embodiment, the present invention is also directed to a composition as disclosed above, wherein the aerogel particles have a bulk density in the range of from 0.03 to 1 kg/m³, for example in the range of from 0.03 to 0.5 kg/m³.

Furthermore, suitable aerogel particles preferably have a degree of crosslinking in the range of from 0.5 to 2, more preferable in the range of from 0.75 to 1.75, in particular in the range of from 1.0 to 1.5.

Therefore, according to a further embodiment, the present invention is also directed to a composition as disclosed above, wherein the aerogel has a degree of crosslinking in the range of from 0.5 to 2.

According to the present invention, the aerogel particles comprise an alginate. The aerogel particles also can comprise further components, preferably components which form a stable structure with the alginate. Suitable further components are for example polysaccharides. Suitable polysaccharides are for example natural polysaccharides such as agar, carrageenan, cellulose, hyaluronic acid, pectin, starch, and xanthan gum as well as semi-synthetic polysaccharides such as modified cellulose, chitin and chitosan.

Therefore, according to a further embodiment, the present invention is also directed to a composition as disclosed above, wherein the aerogel comprises one or more further polysaccharides.

It was found that a particular suitable aerogel comprising an alginate is an aerogel prepared according to the process as disclosed in WO 2015/177081 A1. The aerogels obtained are particularly suitable. Therefore, according to a further embodiment, the present invention is also directed to a composition as disclosed above, wherein the aerogel is obtained or obtainable by a process for preparing a porous material, at least comprising the steps of:

a) providing a mixture (I) comprising
(i) a water-soluble alginate,
(ii) at least one compound suitable to react as cross-linker for the alginate or to release a cross-linker for the alginate,
(iii) water,
   b) preparing a gel (A) comprising steps b1) and b2)
b1) exposing mixture (I) to carbon dioxide at a pressure in the range of from 20 to 100 bar for a time sufficient to form a gel (A), and
b2) depressurizing the gel (A),
   c) exposing the gel (A) obtained in step b) to a water miscible solvent (L) to obtain a gel (B),
   d) drying of the gel (B) obtained in step c).

It has surprisingly been found that the aerogel obtained or obtainable by the process as set out above are particularly suitable for release or absorption and release of formulation components such as emollients, or active materials.

For the purposes of the present invention, a gel is a crosslinked system based on a polymer which is present in contact with a liquid (known as solvogel or lyogel), or with water as liquid (aquagel or hydrogel). Here, the polymer phase forms a continuous three-dimensional network.

In the context of the present invention, water soluble means that the solubility in water is sufficient to form a solution which can be used for preparing a gel.

The process may also comprise the steps a) to d):

a) providing a mixture (I) comprising
(i) a water soluble alginate,
(ii) at least one compound suitable to react as cross-linker for the alginate or to release a cross-linker for the alginate,
(iii) water,
   b) preparing a gel (A) comprising steps b1) and b2)
b1) exposing mixture (I) to carbon dioxide at a pressure in the range of from 20 to 100 bar for a time sufficient to form a gel (A), and
b2) depressurizing the gel (A),
   c) exposing the gel (A) obtained in step b) to a water miscible solvent (L) to obtain a gel (B),
   d) drying of the gel (B) obtained in step c).

According to this process, a gel is formed from the water soluble algiante and a suitable cross-linker. It is possible to influence the properties of the aerogels by varying the ratio of the components, as well as by pressure control and also by introducing a wide range of organic and inorganic materials in the gel matrix. Both mesoporous and macroporous (foamlike) aerogels can be produced by said process. Furthermore, there are no organic byproducts associated with the process which make the aerogels obtained particularly suitable for cosmetic and/or dermatological applications.

The water-soluble alginates used in step a) preferably are alkali metal alginates such as alginates of sodium, or potassium. The underlying alginic acid is a natural acid polysaccharide primarily extracted from so-called brown algae (Phaecophyceae) with a high molecular weight between 30,000 and 200,000, which contains chains formed from D-mannuronic acid and L-guluronic acid. The degree of polymerization changes depending on the kind of alga used for extraction, on the season during which the algae were collected, the geographic origin of the algae as well as the age of the plants. The main kinds of brown algae from which alginic acid is obtained, are, for example Macrocystis pyrifera, Laminaria cloustoni, Laminaria hyperborea, Laminaria flexicaulis, Laminaria digitata, Ascophyllumnodosum and Fucus serratus. However, alginic acid or alkali alginates can also be obtained microbiologically, for example by fermentation with Pseudomonas aeruginosa or mutants of Pseudomonas putida, Pseudomonas fluorescens or Pseudomonas mendocina, see. e.g. EP-A-251905 and the entry regarding “alginic acid” in Römpp Chemie Lexikon “Naturstoffe” (Encyclopedia of Natural Products) published by Thieme Verlag, 1997.

The aqueous solution of the water-soluble alginate used in step a) preferably has such a concentration, that, in mixture (I) a concentration is formed of 0.2 to 3 wt %, more preferably 0.3 wt % to 2.5 wt %, and still more preferably 0.4 wt % to 1.2 wt % of alginate in relation to the amount of water used. The solution can be prepared by suspending the desired amount of alginate in, e.g., distilled water.

The compound suitable to react as cross-linker for the alginate or to release a cross-linker for the alginate preferably is selected from the group consisting of carbonates and hydroxy carbonates. The solvent (L) can in principle be any suitable compound or mixture of a plurality of compounds, which meets the above requirements with the solvent (L) being liquid under the temperature and pressure conditions of step c).

The mixture (I) provided in step (a) can also comprise further salts, in particular such salts that do not form gels, and customary auxiliaries known to those skilled in the art as further constituents. Mention may be made by way of example of surface-active substances, nucleating agents, oxidation stabilizers, dyes and pigments, stabilizers, e.g. against hydrolysis, light, heat or discoloration, inorganic and/or organic fillers. Furthermore, the mixture (I) can comprise cosmetic or medical active substances.

Step c) might be carried out at ambient pressure. However, it is also possible to carry out step c) at elevated pressure. Preferably, step c) is carried out at a pressure above 10 bar, in particular at a pressure below 150 bar.

In step c), gel (B) is obtained. According to step d) of the process, gel (B) obtained in step c) is dried. Further details regarding the process and preferred embodiments are disclosed in WO 2015/177081 A1.

The aerogel particles according to the invention preferably has a density in the range of 0.005 to 1 g/cm³, preferably from 0.01 to 0.5 g/cm³ (determined according to DIN 53420).

The average pore diameter is determined by scanning electron microscopy and subsequent image analysis using a statistically significant number of pores. Corresponding methods are known to those skilled in the art. For characterization of the porous structure of aerogels a Nova 3000 Surface Area Analyzer from Quantachrome Instruments was used. It uses adsorption and desorption of nitrogen at a constant temperature of −196° C.

The volume average pore diameter of the aerogel is preferably not more than 4 microns. The volume average pore diameter of the porous material is particularly preferably not more than 3 microns, very particularly preferably not more than 2 microns and in particular not more than 1 micron. Therefore, according to a further embodiment, the present invention is also directed to a composition as disclosed above, wherein the volume average pore diameter of the aerogel is not more than 4 microns.

Suitable aerogels have a practical lower limit for the volume average pore diameter. In general, the volume average pore diameter is at least 20 nm, preferably at least 50 nm. Therefore, according to a further embodiment, the present invention is also directed to a composition as disclosed above, wherein the volume average pore diameter of the aerogel particles is at least 20 nm, preferably at least 50 nm.

The aerogel used according to the invention preferably has a porosity of at least 70% by volume, in particular from 70 to 99% by volume, particularly preferably at least 80% by volume, very particularly preferably at least 85% by volume, in particular from 85 to 95% by volume. The porosity in % by volume means that the specified proportion of the total volume of the porous material comprises pores.

Therefore, according to a further embodiment, the present invention is also directed to a composition as disclosed above, wherein the aerogel has a porosity of at least 70% by volume, in particular from 70 to 99% by volume.

According to the present invention, the composition may comprise the aerogel particles in any suitable amount. The aerogel may for example be present in the composition in an amount in the range of from 0.02 to 10% by weight, based on the total weight of the composition, preferably in an amount in the range of from 0.1 to 5% by weight, based on the total weight of the composition, more preferable in an amount in the range of from 0.1 to 0.5% by weight, based on the total weight of the composition.

Therefore, according to a further embodiment, the present invention is also directed to a composition as disclosed above, wherein the aerogel is present in the composition in an amount in the range of from 0.02 to 10% by weight, preferably in the range of from 0.02 to 5% by weight based on the total weight of the composition.

The composition according can comprise further ingredients, in particular suitable carrier materials such as oils and/or waxes.

Mention may be made, as oils which can be used in the composition of the invention, for example, of:
hydrocarbon oils of animal origin, such as perhydrosqualene;
hydrocarbon oils of plant origin, such as liquid triglycerides of fatty acids containing from 4 to 10 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, castor oil, avocado oil, caprylic/capric acid triglycerides, for instance those sold by Stearineries Dubois or those sold under the names Miglyol 810, 812 and 818 by Dynamit Nobel, jojoba oil and shea butter oil; and
esters of plant origin;
synthetic esters and ethers, especially of fatty acids, for instance Purcellin oil, isononyl isononanoate, isopropyl myristate, 2-ethylhexyl palmitate, 2-octyldodecyl stearate, 2-octyldodecyl erucate or isostearyl isostearate; hydroxylated esters, for instance isostearyl lactate, octyl hydroxystearate, octyldodecyl hydroxystearate, diisostearyl malate or triisocetyl citrate; fatty alcohol heptanoates, octanoates or decanoates; polyol esters, for instance propylene glycol dioctanoate, neopentyl glycol diheptanoate and diethylene glycol diisononanoate; and pentaerythritol esters, for instance pentaerythrityl tetraisostearate;
straight or branched hydrocarbons of inorganic or synthetic origin, such as volatile or non-volatile liquid paraffins, and derivatives thereof, petroleum jelly, polydecenes, alkanes and hydrogenated polyisobutene such as Parleam oil;
fatty alcohols having from 8 to 26 carbon atoms, such as cetyl alcohol, stearyl alcohol and their mixture (cetearyl alcohol), octyldodecanol, 2-butyloctanol, 2-hexyldecanol, 2-undecylpentadecanol, oleyl alcohol or linoleyl alcohol;
partially hydrocarbon-based and/or silicone-based fluoro oils;
silicone oils, for instance volatile or non-volatile polymethylsiloxanes (PDMSs) with a straight or cyclic silicone chain, which are liquid or pasty at room temperature, especially cyclopolydimethylsiloxanes (cyclomethicones) such as cyclohexasiloxane; polydimethylsiloxanes containing alkyl, alkoxy or phenyl groups, which are pendent or at the end of a silicone chain, these groups containing from 2 to 24 carbon atoms; phenyl silicones, for instance phenyl trimethicones, phenyl dimethicones, phenyltrimethylsiloxydiphenylsiloxanes, diphenyl dimethicones, diphenylmethyldiphenyltrisiloxanes or 2-phenylethyl trimethylsiloxy silicates, and polymethylphenylsiloxanes;
mixtures thereof.

In particular, said oils are selected from the group consisting of mineral oils; paraffin oils; vaseline; linear saturated hydrocarbons, preferably having more than 8 carbon atoms, such as tetradecane, hexadecane, octadecane and polydecene; branched hydrocarbons like hydrogenated polyisobutene, squalane and squalene, cyclic hydrocarbons, such as decahydronaphthalene cycloparaffin, animal and vegetable oils, synthetic or semisynthetic oils, silicone oils.

Suitable silicone oils are linear polydimethylsiloxanes, poly(methylphenylsiloxanes), cyclic siloxanes and mixtures thereof. The number-average molecular weight of the polydimethylsiloxanes and poly(methylphenylsiloxanes) is preferably in a range from about 1000 to 150 000 g/mol. Preferred cyclic siloxanes have 4- to 8-membered rings. Suitable cyclic siloxanes are commercially available, for example, under the name Cyclomethicone®.

Animal and vegetable oils are selected from the group of natural fats and oils, such as rapeseed oil, castor oil, soybean oil, wheat germ oil, peanut oil, macadamia nut oil, olive oil, sunflower oil, sesame oil, jojoba oil, avocado oil, cocoa butter, almond oil, palm oil, coconut oil, grape seed oil, thistle oil, evening primrose oil, persic oil, ricinus oil, cod liver oil, lard, spermaceti, spermaceti oil, sperm oil. Said animal and vegetable oils are also selected from essential oils of lower volatility, which are mostly used as aroma components or perfume oils. In particular said oils are chosen from the group of sage oil, clary sage oil, camomile oil, oil of cloves, melissa oil, mint oil, eucalyptus oil, cinnamon leaf oil, linden blossom oil, juniper berry oil, vetiver oil, olibanum oil, galbanum oil, labolanum oil, rose hip oil bergamot oil, lemon oil, mandarin oil, orange oil and lavandin oil.

Semisynthetic oils are selected from the group of dihydromyrcenol, lilial, lyral, citronellol, phenylethyl alcohol, alpha-hexylcinnamaldehyde, geraniol, benzyl acetone, cyclamenaldehyde, linalool, boisambrene forte, ambroxan, indole, hedione, sandelice, allyl amyl glycolate, cyclovertal, D-damascone, geranium oil bourbon, cyclohexyl salicylate, vertofix coeur, iso-E-super, Fixolide NP, evernyl, iraldein gamma, phenylacetic acid, geranyl acetate, benzyl acetate, rose oxide, rommilat, irotyl and floramat.

Fats are also understood to be comprised within the cosmetically acceptable carrier as mentioned. Said fats comprise the group of lecithins and fatty acid triglycerides, namely the triglycerol esters of saturated and/or unsaturated, branched and/or unbranched alkanecarboxylic acids with a chain length of from 8 to 24, in particular 12 to 18, carbon atoms. One source of fatty acid triglycerides are synthetic, semisynthetic or natural oils comprising the animal and vegetable oils given supra.

Fats do also comprise chemically modified fats, such as, for instance, hydrogenated vegetable oils like hydrogenated castor oil and/or hydrogenated coconut fatty glycerides, triglycerides, such as, hydrogenated soy glyceride, trihydroxystearin, shea butter, uropygial grease.

Fats frequently used are caprylic/capric triglycerides. Said waxes of said cosmetically acceptable carrier are compounds which at 20° C. are solid to fragile, are susceptible to be kneaded and have a rough to compact grained structure. Their appearance is translucent to opaque, but not vitreous. They melt at temperatures above 40° C. without decomposition and just above said temperature, they are liquid and barely viscous. Their consistency and solubility considerably depends on temperature. Said compounds are susceptible to be polished under moderate pressure.

Typical waxes, liquid or solid, are selected from the group comprising esters of saturated and/or unsaturated, branched and/or unbranched alkanecarboxylic acids with a chain length from 3 to 30 carbon atoms and saturated and/or unsaturated, branched and/or unbranched alcohols with a chain length of from 3 to 30 carbon atoms as well as from the group of esters of aromatic carboxylic acids and saturated and/or unsaturated, branched and/or unbranched alcohols with a chain length of from 3 to 30 carbon atoms. More preference is given to esters of C1-C24-monoalcohols with C1-C22-monocarboxylic acids like for instance C20-C40-alkyl stearate, C20-C40-alkyl hydroxystearoyl stearate.

In a preferred embodiment the wax is selected from the group of isononyl isononanoate, isotridecyl isononanoate, n-hexyl laurate, 2-ethylhexyl laurate, n-propyl myristate, isopropyl myristate, n-propyl palmitate, 2 ethylhexyl palmitate, isopropyl palmitate, hexacosanyl palmitate, 2-octyldodecyl palmitate, octacosanyl palmitate, cetyl palmitate, triacontanyl palmitate, dotriacontanyl palmitate, tetratriacontanyl palmitate, n-butyl stearate, isopropyl stearate, isopropyl isostearate, isooctyl stearate, isononyl stearate, 2-hexyldecyl stearate, 2-ethylhexyl isostearate, hexacosanyl stearate, octacosanyl stearate, triacontanyl stearate, dotriacontanyl stearate, tetratriacontanyl stearate, isopropyl oleate, n-decyl oleate, oleyl oleate, oleyl erucate, erucyl oleate, erucyl erucate, dicaprylyl carbonate (Cetiol CC) dibutyl adipate, 2-ethylhexyl cocoate, cocoglycerides (Myritol 331), glycol esters, such as butylene glycol dicaprylate/dicaprate, dicaprylyl ether, propylene glycol monolaurate, polyethylene glycol monolaurate, glycol montanate, octyldodecanol, isoeicosane, C10-C16-alkylbenzoate, benzyl benzoate, C1-C10-salicylates, e.g. octyl salicylate, C10-C16-alkyl lactates or from mixtures thereof.

In a further preferred embodiment the wax is selected from the group of mixtures of C12-C16-alkyl benzoate and 2-ethylhexyl isostearate, mixtures of C12-C16-alkyl benzoate, 2-ethylhexyl isostearate and isotridecyl isononanoate, mixtures of C12-C16-alkyl benzoate and isotridecyl isononanoate, mixtures of C12-C16-alkyl benzoate, 2-ethylhexyl isostearate and isotridecyl isononanoate, mixtures of octyldodecanol, dicaprylyl ether, dicaprylyl carbonate, cocoglycerides, mixtures of C12-C16-alkyl benzoate and butylene glycol dicaprylate/dicaprate.

Preferred waxes from the group of vegetable waxes, animal waxes, mineral waxes and petrochemical waxes used within the cosmetically acceptable carrier B are beeswax, berry wax, carnauba wax, candelilla wax, ceresine, cork wax, esparto grass wax, guaruma wax, japan wax, jojoba wax, lanolin (wool wax), microwaxes, montan wax, ouricury wax, ozokerite (earth wax), paraffin waxes, ricegerm oil wax, shellac wax, spermaceti, sugarcane wax and mixtures of the abovementioned compounds.

The wax of the cosmetically acceptable carrier is also selected from chemically modified waxes and synthetic waxes, such as Syncrowax® HRC (glyceryl tribehenate), Syncrowax® AW 1 C (C16-C36-fatty acid) and montan ester waxes, sasol waxes, hydrogenated jojoba waxes, synthetic or modified beeswaxes (e.g. dimethicone copolyol beeswax and/or C30-C50-alkyl beeswax), cetyl ricinoleates, such as, e.g., Tegosoft® CR, polyalkylene waxes, polyethylene glycol waxes.

The cosmetically acceptable carrier also comprises at least one fatty acid, which is selected from the group of myristic acid, stearic acid, palmitic acid, oleic acid, linoleic acid, linolenic acid as well as from saturated, unsaturated and substituted modifications thereof.

In another embodiment the carrier is at least one fatty alcohol selected from the group of lauryl alcohol, myristyl alcohol, cetyl alcohol, stearyl alcohol, oleyl alcohol, cetearyl alcohol, 2-butyloctanol (available commercially, for example, as Isofol®12 (Condea)), 2-hexyldecanol (available commercially, for example, as Isofol®16 (Condea)).

According to one particular embodiment of the invention, the composition according to the invention is a water-in-oil (W/O) or oil-in-water (O/W) emulsion, preferably an O/W emulsion. The O/W emulsion also comprises emulsified gels. The term “emulsified gels” means dispersions of oils in an aqueous gel. The addition of surfactant is optional for this pharmaceutical form.

The proportion of the oil phase of the emulsion may range from 2% to 80% by weight and preferably from 5% to 50% by weight relative to the total weight of the composition.

The emulsions generally contain at least one emulsifier chosen from amphoteric, anionic, cationic or non-ionic emulsifiers, used alone or as a mixture, and optionally a co-emulsifier. The emulsifiers are appropriately chosen according to the emulsion to be obtained (W/0 or 0/W). The emulsifier and the co-emulsifier are generally present in the composition in a proportion ranging from 0.05% to 30% by weight and preferably from 0.1% to 20% by weight relative to the total weight of the composition.

The composition may be an aqueous gel and may especially comprise common aqueous gelling agents. Advantageously, the composition is a composition comprising at least one aqueous phase. The aqueous phase generally represents more than 5% by weight of the total weight of the composition and preferably more than 20% by weight.

The composition according to the invention comprises an aqueous phase comprising water and optionally glycols, ethanol and/or hydrophilic adjuvants, which may be water-soluble at room temperature. The composition generally comprises from 30 to 95% water.

Therefore, according to a further embodiment, the present invention is also directed to a composition as disclosed above, wherein the composition comprises at least one aqueous phase.

Advantageously, the composition according to the invention has a pH ranging from 3 to 8. Preferably, the pH of the composition ranges from 4 to 7. The composition according to the invention may be a composition for caring for, cleansing or making up bodily or facial skin, in particular a care composition. The skincare composition may be, for example, a facial cream, gel or fluid.

The composition according to the present invention may also contain various adjuvants commonly used in cosmetics, such as emulsifiers; fillers; preserving agents; sequestrants; dyes; fragrances; thickeners and gelling agents, in particular acrylamide homopolymers and copolymers, acrylic homopolymers and copolymers and acrylamidomethylpropanesulfonic acid (AMPS®) homopolymers and copolymers as well as polyurethanes, polyvinylpyrrolidones and vinyl pyrrolidone copolymers, styrene acrylate copolymers, Vinyl pyrrolidone, vinyl acetate copolymers; UV-screening agents, chelating agents, buffers, pigments

The composition may also contain cosmetic active agents, for instance moisturizers and vitamins. Naturally, a person skilled in the art will take care to select this or these optional additional compound(s), and/or the amount thereof, such that the advantageous properties of the composition according to the invention are not, or are not substantially, adversely affected by the envisaged addition.

Therefore, according to a further embodiment, the present invention is also directed to a composition as disclosed above, wherein the composition further comprises suitable additives.

Depending on their nature and the purpose of the composition, the standard cosmetic ingredients can be present in standard amounts which can be easily determined by those skilled in the art and which can be, generally, for each ingredient, between 0.01% and 80% by weight.

A person skilled in the art will take care to choose the ingredients included in the composition, and also the amounts thereof, such that they do not harm the properties of the compositions of the present invention.

Suitable methods for preparing the composition according to the present invention are in principle known to the person skilled in the art.

The cosmetic composition according to the invention may be in the form of a product for caring for, cleansing and/or making up bodily or facial skin, the lips, the eyebrows, the eyelashes, the nails and the hair, an antisun or self-tanning product, a body hygiene product, or a haircare product, especially for caring for, cleansing, styling, shaping or coloring the hair.

According to a further aspect, the present invention is also directed to the cosmetic use of the composition as disclosed above, for treating the skin. In particular, the present invention is directed to the cosmetic use of a cosmetic and/or dermatological composition comprising, in a physiologically acceptable medium aerogel particles comprising an alginate, for treating the skin.

Preferred embodiments may be found in the claims and the description. Combinations of preferred embodiments do not go outside the scope of the present invention. Preferred embodiments of the components used are described below.

The present invention includes the following embodiments, wherein these include the specific combinations of embodiments as indicated by the respective interdependencies defined therein.
1. Cosmetic and/or dermatological composition comprising, in a physiologically acceptable medium aerogel particles comprising an alginate.
2. The composition according to embodiment 1, wherein the aerogel particles have a bulk density in the range of from 0.03 to 1 kg/m³.
3. The composition according to embodiment 1 or 2, wherein the aerogel has a degree of crosslinking in the range of from 0.5 to 2.
4. The composition according to any one of embodiments 1 to 3, wherein the aerogel comprises one or more further polysaccharides.
5. The composition according to any one of embodiments 1 to 4, wherein the aerogel is obtained or obtainable by a process for preparing a porous material, at least comprising the steps of:
   a) providing a mixture (I) comprising
      (i) a water soluble alginate,
      (ii) at least one compound suitable to react as cross-linker for the alginate or to release a cross-linker for the alginate,
      (iii) water,
   b) preparing a gel (A) comprising steps b1) and b2)
   b1) exposing mixture (I) to carbon dioxide at a pressure in the range of from 20 to 100 bar for a time sufficient to form a gel (A), and
   b2) depressurizing the gel (A),
   c) exposing the gel (A) obtained in step b) to a water miscible solvent (L) to obtain a gel (B),
   d) drying of the gel (B) obtained in step c).
6. The composition according to any one of embodiments 1 to 5, wherein the volume average pore diameter of the aerogel is not more than 4 microns.
7. The composition according to any one of embodiments 1 to 6, wherein the volume average pore diameter of the aerogel particles is at least 20 nm, preferably at least 50 nm.
8. The composition according to any one of embodiments 1 to 7, wherein the aerogel has a porosity of at least 70% by volume, in particular from 70 to 99% by volume.
9. The composition according to any one of embodiments 1 to 8, wherein the aerogel is present in the composition in an amount in the range of from 0.02 to 10% by weight, preferably in the range of from 0.02 to 0.5% by weight, based on the total weight of the composition.
10. The composition according to any one of embodiments 1 to 9, wherein the composition comprises at least one aqueous phase.
11. Cosmetic and/or dermatological composition comprising, in a physiologically acceptable medium aerogel particles comprising an alginate, and
   wherein the aerogel particles have a bulk density in the range of from 0.03 to 1 kg/m³.
12. Cosmetic and/or dermatological composition comprising, in a physiologically acceptable medium aerogel particles comprising an alginate, and
   wherein the aerogel has a degree of crosslinking in the range of from 0.5 to 2.
13. Cosmetic and/or dermatological composition comprising, in a physiologically acceptable medium aerogel particles comprising an alginate,
   wherein the aerogel particles have a bulk density in the range of from 0.03 to 1 kg/m³, and
   wherein the aerogel has a degree of crosslinking in the range of from 0.5 to 2.
14. Cosmetic and/or dermatological composition comprising, in a physiologically acceptable medium aerogel particles comprising an alginate, and
   wherein the aerogel comprises one or more further polysaccharides.
15. Cosmetic and/or dermatological composition comprising, in a physiologically acceptable medium aerogel particles comprising an alginate, and
   wherein the aerogel is obtained or obtainable by a process for preparing a porous material, at least comprising the steps of:
   a) providing a mixture (I) comprising
      (i) a water soluble alginate,
      (ii) at least one compound suitable to react as cross-linker for the alginate or to release a cross-linker for the alginate,
      (iii) water,
   b) preparing a gel (A) comprising steps b1) and b2)
   b1) exposing mixture (I) to carbon dioxide at a pressure in the range of from 20 to 100 bar for a time sufficient to form a gel (A), and
   b2) depressurizing the gel (A),
   c) exposing the gel (A) obtained in step b) to a water miscible solvent (L) to obtain a gel (B),
   d) drying of the gel (B) obtained in step c).
16. Cosmetic and/or dermatological composition comprising, in a physiologically acceptable medium aerogel particles comprising an alginate,
   wherein the volume average pore diameter of the aerogel is not more than 4 microns, and
   wherein the volume average pore diameter of the aerogel particles is at least 20 nm, preferably at least 50 nm.
17. Cosmetic and/or dermatological composition comprising, in a physiologically acceptable medium aerogel particles comprising an alginate, wherein the aerogel is obtained or obtainable by a process for preparing a porous material, at least comprising the steps of:
   a) providing a mixture (I) comprising
      (i) a water soluble alginate,
      (ii) at least one compound suitable to react as cross-linker for the alginate or to release a cross-linker for the alginate,
      (iii) water,
   b) preparing a gel (A) comprising steps b1) and b2)
   b1) exposing mixture (I) to carbon dioxide at a pressure in the range of from 20 to 100 bar for a time sufficient to form a gel (A), and
   b2) depressurizing the gel (A),
   c) exposing the gel (A) obtained in step b) to a water miscible solvent (L) to obtain a gel (B),
   d) drying of the gel (B) obtained in step c),
   wherein the volume average pore diameter of the aerogel is not more than 4 microns, and
   wherein the volume average pore diameter of the aerogel particles is at least 20 nm, preferably at least 50 nm.
18. Cosmetic and/or dermatological composition comprising, in a physiologically acceptable medium aerogel particles comprising an alginate, and
   wherein the aerogel has a porosity of at least 70% by volume, in particular from 70 to 99% by volume.
19. Cosmetic and/or dermatological composition comprising, in a physiologically acceptable medium aerogel particles comprising an alginate,
   wherein the volume average pore diameter of the aerogel is not more than 4 microns, wherein the volume average pore diameter of the aerogel particles is at least 20 nm, preferably at least 50 nm, and
   wherein the aerogel has a porosity of at least 70% by volume, in particular from 70 to 99% by volume.
20. Cosmetic use of the composition according to any one of embodiments 1 to 17, for treating the skin.

Examples will be used below to illustrate the invention.

### EXAMPLES

1. Preparation of Water Soluble Films from Aerogel

### 1.1 Cold Process

The required amount of Aerogel will be given to VE-water while stirring. The aerogels directly start swelling. After 5 min. of additional stirring, a homogenization with an Ultra Turax is recommended. The ph-value can be adjusted as required with citric acid or another cosmetically acceptable pH adjusting agent.

The prepared aqueous Aerogel will be now poured out of a smooth surface in the required thickness. After drying at low temperatures, strong inflexible films are formed.

With water contact the product starts to swell again and return to gel.

TABLE 1
Basic test formulation for skin care

| | | | % by | |
|---|---|---|---|---|
| Phase | Ingredients | INCI | weight | Function |
| A | Eumulgin ® SG | Sodium Stearoyl | 1.00 | Emulsifier |
| | | Glutamate | | (O/W) |
| | Cutina HVG | Hydrogenated | 5.00 | Consistency |
| | | Vegetable Glycerides | | agent |
| | Cetiol ® LC | Coco-Caprylate/Caprate | 16.00 | Emollient |
| B | Aerogel | Ca-Alginat | 0.75 | |
| | Water, demin. | | 78.00 | |
| C | Euxyl K 320 | | 1.00 | Preservative |
| | citric acid | | q.s. | pH adjustment |

### 1.2 Manufacturing Process

Heat phase A and phase B separately up to 85° C. Add phase B to phase A while stirring.

Stir in such a way that it remains in continual motion. Avoid the incorporation of air. Homogenize at 55° C. by means of a suitable dispersion unit (e.g. Ultra Turrax) in order to improve stability and structure.

Cool to 40° C. and add components of phase C one by one while stirring.

Finally adjust the pH-value to approx. 6.5. Stop stirring at 30° C. to give a smooth emulsion with a Brookfield viscosity of 65000 mPa*s measured with Brookfield Helipath and spindle 94 at 4 rpm.

| | |
|---|---|
| Eumulgin SG | 1.0 |
| Cutina HVG | 1.0 |
| Lanette O | 3.0 |
| Myritol 331 | 6.0 |
| Cetiol LC | 3.0 |
| preservative | q.s. |
| Glycerin | 5.0 |
| Water | ad 100 |

TABLE 2a

| Reference | −5 | −1 | −2 | −3 |
|---|---|---|---|---|
| Variation | Placebo | 1% . . . 13 | 0.75% . . . 14 | 1.0% . . . 13 |
| | | Oil phase | Oil phase | Water phase |
| Viscosity | 62.500 | 137.500 | 75.000 | 162.500 |
| (mPas) | | | | |
| (Brookfield | | | | |
| RVF, TE-spindle, | | | | |
| 4 rpm, Helipath) | | | | |
| pH-value | 7.4 | 7.2 | 7.6 | 7.1 |
| Macro | B | C | B | C |
| Micro | Evenly | Partly | Evenly | Evenly |
| | homogeneous, | crystalline | homogeneous | homogeneous |
| | relatively | structures, | | |
| | large oil drops | relatively | | |
| | | evenly | | |
| appearance | foamy | slimy/ | smooth | slimy, like |
| | | inhomogeneous | | GTC |
| Stability/ | RT and −5° C. | Do not look | −5° C., RT, | 40° C. |
| stabel | | good | 40° C. | |
| 12 weeks | | | | |
| Stability/ | 4 weeks, | 4 weeks, | 8 weeks, | 12 weeks, |
| separated | 45° C. | 50° C. | 50° C. | 50° C. |
| | 8 weeks, | | | |
| | 40, 50° C. | | | |
| viscosity after | 37.500 | 137.500 | 100.000 | 187.500 |
| 12 weeks/RT (mPas) | | | | |
| (Brookfield RVF, | | | | |
| TE-spindle, 4 rpm, | | | | |
| Helipath) | | | | |

TABLE 2b

| Reference | −4 | −6 | −7 | −8 | −9 |
|---|---|---|---|---|---|
| Variation | 1.0% . . . 14 | 0.3% . . . 13 | 2.0% . . . 13 | 0.3% . . . 14 | 2.0% . . . 14 |
| | water phase | water phase | water phase | Water phase | Water phase |
| Viscosity | 175.000 | 137.500 | 325.000 | 112.500 | 300.000 |
| (mPas) | | | | | |
| (Brookfield | | | | | |
| RVF, TE- | | | | | |
| spindle, 4 | | | | | |
| rpm, Helipath) | | | | | |
| pH-value | 7.5 | 7.6 | 7.6 | 7.8 | 7.9 |
| Macro | C | B-C | C | B-C | C |
| Micro | Evenly | Evenly | Lamellar | Evenly | Lamellar |
| | homogeneous | homogeneous | structures, | homogeneous | structures, |
| | | | large and | | large and |
| | | | uneven | | uneven |
| appearance | slimy, like | smooth | extremely | smooth | extremely |
| | GTC | | ,,griesig“ | | ,,griesig“ |
| Stability/ | −5° C. | −5° C., RT, | Not stored | −5° C., RT, | Not |
| stabel 12 | | 40° C. | | 40° C. | stored |
| weeks | | | | | |
| Stability/ | 12 weeks, | 2 weeks, | | 2 weeks, | |
| separated | 50° C. | 50° C. | | 50° C. | |
| | | 12 weeks, | | 4 weeks, | |
| | | 45° C. | | 45° C. | |
| viscosity after | 200.000 | 87.500 | | 87.500 | |
| 12 weeks/RT | | | | | |
| (mPas) | | | | | |
| (Brookfield | | | | | |
| RVF, TE- | | | | | |
| spindle, 4 | | | | | |
| rpm, Helipath) | | | | | |

### LITERATURE CITED

WO 2013/190104 A2
US 2015/0190319 A1
WO 2015/177081 A1
EP-A-251905
“Alginic Acid” in Rompp Chemie Lexikon “Naturstoffe” (Encyclopedia of Natural Products), Thieme Verlag, 1997
WO 2015/177081 A1

1. A cosmetic and/or dermatological composition comprising, in a physiologically acceptable medium, aerogel particles comprising an alginate.

2. The composition according to claim 1, wherein the aerogel particles have a bulk density in the range of from 0.03 to 1 kg/m³.

3. The composition according to claim 1, wherein the aerogel has a degree of cross-linking in the range of from 0.5 to 2.

4. The composition according to claim 1, wherein the aerogel comprises one or more further polysaccharide.

5. The composition according to claim 1, wherein the aerogel is obtained by a process for preparing a porous material, comprising:
a) providing a mixture (I) comprising
(i) a water soluble alginate,
(ii) at least one compound suitable to react as cross-linker for the alginate or to release a cross-linker for the alginate,
(iii) water,
b) preparing a gel (A) comprising steps b1) and b2)
b1) exposing mixture (I) to carbon dioxide at a pressure in the range of from 20 to 100 bar for a time sufficient to form a gel (A), and
b2) depressurizing the gel (A),
c) exposing the gel (A) obtained in step b) to a water miscible solvent (L) to obtain a gel (B),
d) drying of the gel (B) obtained in step c).

6. The composition according to claim 1, wherein a volume average pore diameter of the aerogel is not more than 4 microns.

7. The composition according to claim 1, wherein a volume average pore diameter of the aerogel particles is at least 20 nm.

8. The composition according to claim 1, wherein the aerogel has a porosity of at least 70% by volume.

9. The composition according to claim 1, wherein the aerogel is present in the composition in an amount in the range of from 0.02 to 10% by weight, based on the total weight of the composition.

10. The composition according to claim 1, wherein the composition comprises at least one aqueous phase.

11. The composition according to claim 1, for use in treating the skin.

12. The composition according to claim 7, wherein the average pore diameter of the aerogel particles is at least 50 nm.

13. The composition according to claim 8, wherein the aerogel has a porosity of 70 to 99% by volume.

14. The composition according to claim 9, wherein the aerogel is present in the composition in an amount from 0.02 to 0.5% by weight-based on the total weight of the composition.
